(19) United States
(12) Patent Application Publication
(10) Pub. No.: US 2018/0197635 A1
(43) Pub. Date: Jul. 12, 2018

(54) SYSTEM AND METHOD FOR DETERMINING USER HEALTH

(71) Applicant: Machine Zone, Inc., Palo Alto, CA (US)

(72) Inventors: Nikhil Bojja, Mountain View, CA (US); Michael Fox, Pacifica, CA (US); Nathan Spencer, Redwood City, CA (US); Calvin Lau, Newark, CA (US); David Severs, Palo Alto, CA (US); Andrew Koike, San Jose, CA (US); Shiman Guo, Cupertino, CA (US)

(21) Appl. No.: 15/795,946

(22) Filed: Oct. 27, 2017

Related U.S. Application Data
(60) Provisional application No. 62/444,476, filed on Jan. 10, 2017.

(51) Int. Cl. G16H 50/20; G06N 5/02; G06N 99/00

(52) U.S. Cl. CPC G16H 50/20; G06N 99/005; G06N 5/022

(57) ABSTRACT

A method, a system, and an article are provided for determining how active users and groups of users are in an online game and, based thereon, generating recommendations for users to join one or more of the groups. The method can include, for example, generating a representation of a health of each of a plurality of users of a virtual environment, and aggregating the user health representations to generate an aggregated health representation for each group. Based on the aggregated health representations, a recommendation to a selected user of the virtual environment can be generated for joining a recommended group from the plurality of groups.

### CROSS-REFERENCE TO RELATED APPLICATIONS

This application claims the benefit of U.S. Provisional Patent Application No. 62/444,476, filed Jan. 10, 2017, the entire contents of which are incorporated by reference herein.

### BACKGROUND

The present disclosure relates to multi-player online games and, in particular, to systems and methods for determining a health of players and groups of players in the multi-player online game.

In general, a multi-player online game can be played by hundreds of thousands or even millions of players who use client devices to interact with a virtual environment for the online game. The players are typically working to accomplish tasks, acquire assets, or achieve a certain score in the online game. Some games require or encourage players to form groups or teams that can play against other players or groups of players.

In general, player enjoyment in an online game can depend on how engaged other players are in the game. For example, when a player interacts with other players who are highly engaged or involved in the game, the player's experience with the game can be more enjoyable. On the other hand, if a player is interacting with players who are not actively participating in the game, the player is more likely to lose interest in the game.

### SUMMARY

Implementations of the systems and methods described herein can be used to encourage a player of an online game to join a group of players who are actively participating in the online game. In certain examples, player health scores are calculated that provide an indication of how active each player is in the online game. Group health scores can be calculated (e.g., based on the player health scores) that provide an indication of how active each group of players is in the online game. Based on the group health scores, recommendations can be provided to players to join certain groups, for example, the groups with the highest group health scores. Advantageously, by calculating the player health scores and/or the group health scores and providing the recommendations, players are able to become associated with more active players. This improves player satisfaction and generally increases a level of player engagement with the online game.

In one aspect, the subject matter described in this specification relates to a computer-implemented method. The method includes: generating a representation of a health of each of a plurality of users of a virtual environment, wherein each health representation is based on a plurality of metrics generated for a respective user from a history of interactions of the respective user with the virtual environment, and wherein each user is associated with one of a plurality of groups of users within the virtual environment; aggregating the health representations of the users of each group to generate an aggregated health representation for each group; and generating, based on the aggregated health representations, a recommendation to a selected user of the virtual environment for joining a recommended group from the plurality of groups.

In certain examples, the health representation of each user provides an indication of how active the user is within the virtual environment. Generating the health representation can include providing the plurality of metrics to a predictive model including at least one of a trained classifier and a regression model. Generating the health representations can also include: transforming output from the predictive model to achieve a rebalancing of the health representations. In some instances, the plurality of metrics includes at least one of user login activity, user chat activity, user purchasing activity, and any combination thereof. Aggregating the health representations can include determining at least one of an average, a median, a maximum, and a minimum of the health representations of the users of each group.

In various implementations, generating the recommendation includes determining that the recommended group includes an aggregated health representation that exceeds the aggregated health representations of other groups within the plurality of groups. The recommended group can include an aggregated health representation that is a maximum of the aggregated health representations for the plurality of groups. Alternatively or additionally, generating the recommendation can include matching a language preference of the selected user with a language preference of the recommended group. Generating the recommendation can include determining that the recommended group can accommodate the selected user.

In another aspect, the subject matter described in this specification relates to a system that includes one or more computer processors programmed to perform operations including: generating a representation of a health of each of a plurality of users of a virtual environment, wherein each health representation is based on a plurality of metrics generated for a respective user from a history of interactions of the respective user with the virtual environment, and wherein each user is associated with one of a plurality of groups of users within the virtual environment; aggregating the health representations of the users of each group to generate an aggregated health representation for each group; and generating, based on the aggregated health representations, a recommendation to a selected user of the virtual environment for joining a recommended group from the plurality of groups.

In certain examples, the health representation of each user provides an indication of how active the user is within the virtual environment. Generating the health representation can include providing the plurality of metrics to a predictive model including at least one of a trained classifier and a regression model. Generating the health representations can also include transforming output from the predictive model to achieve a rebalancing of the health representations. The plurality of metrics can include at least one of user login activity, user chat activity, user purchasing activity, and any combination thereof.

In various instances, aggregating the health representations can include determining at least one of an average, a median, a maximum, and a minimum of the health representations of the users of each group. Generating the recommendation can include determining that the recommended group includes an aggregated health representation that exceeds the aggregated health representations of other groups within the plurality of groups. The recommended group can include an aggregated health representation that is a maximum of the aggregated health representations for the plurality of groups. Generating the recommendation can include matching a language preference of the selected user with a language preference of the recommended group.

In another aspect, the subject matter described in this specification relates to an article that includes a non-transitory computer-readable medium having executable instructions. The executable instructions are executable by one or more computer processors to perform operations including: generating a representation of a health of each of a plurality of users of a virtual environment, wherein each health representation is based on a plurality of metrics generated for a respective user from a history of interactions of the respective user with the virtual environment, and wherein each user is associated with one of a plurality of groups of users within the virtual environment; aggregating the health representations of the users of each group to generate an aggregated health representation for each group; and generating, based on the aggregated health representations, a recommendation to a selected user of the virtual environment for joining a recommended group from the plurality of groups.

Elements of embodiments described with respect to a given aspect of the invention can be used in various embodiments of another aspect of the invention. For example, it is contemplated that features of dependent claims depending from one independent claim can be used in apparatus, systems, and/or methods of any of the other independent claims

### DESCRIPTION OF THE DRAWINGS

FIG. 1 is a schematic diagram of an example system for determining player health and group health and providing group recommendations to players.

FIG. 2 is a schematic diagram of three groups of users interacting with a virtual environment in an online game, in accordance with certain examples of this disclosure.

FIG. 3 is a flowchart of an example method of providing group recommendations to players.

### DETAILED DESCRIPTION

In general, the systems and methods described herein can be used to determine a health of players and groups of players in an online game. In certain examples, player health is a measure of how active a player is in the game and group health is a measure of how active a group of players is in the game. By determining player health and group health, the systems and methods described herein are able to identify players and groups of players that are active. Other players can then be directed or encouraged to join the active groups and/or leave inactive groups. By connecting players with active groups, overall player enjoyment and satisfaction with the game can be enhanced. In certain instances, a “player” may alternatively be referred to herein as a “user.” A “group” may alternatively be referred to herein as a “team” or an “alliance.” A group of players generally works together to accomplish or reach a common goal and is typically competing with other users or groups of users in the online game.

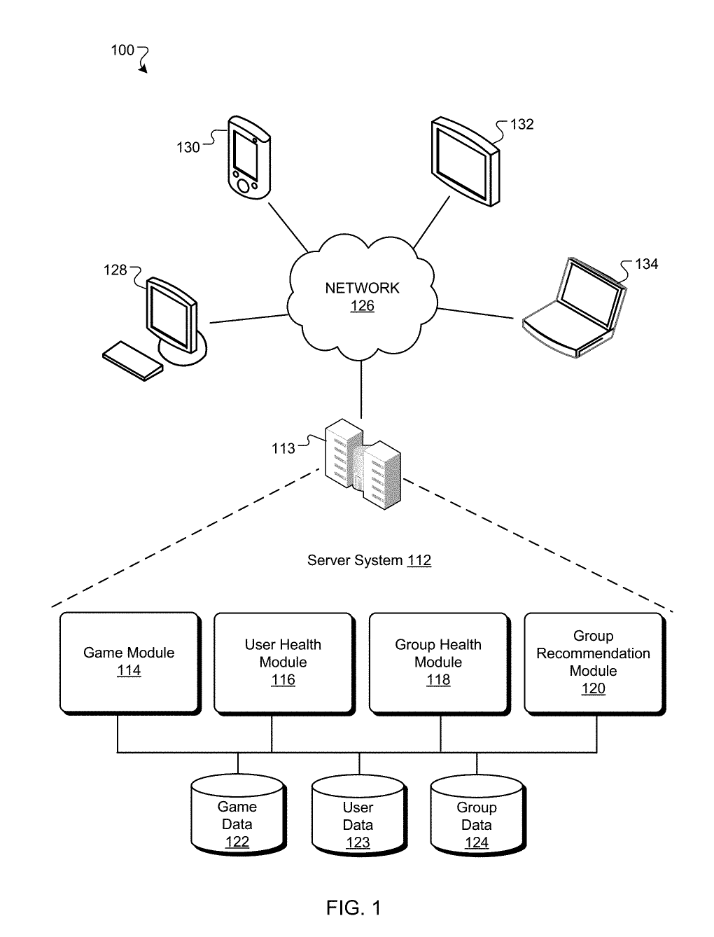

FIG. 1 illustrates an example system 100 for determining player health and group health and encouraging players to join healthy groups in an online game. A server system 112 provides functionality for calculating player and group health scores and generating group recommendations. The server system 112 includes software components and databases that can be deployed at one or more data centers 113 in one or more geographic locations, for example. The server system 112 software components can include a game module 114, a user health module 116, a group health module 118, and a group recommendation module 120. The software components can include subcomponents that can execute on the same or on different individual data processing apparatus. The server system 112 databases can include game data 122, user data 123 and group data 124. The databases can reside in one or more physical storage systems. The software components and data will be further described below.

An application, such as, for example, a web-based application, can be provided as an end-user application to allow users to interact with the server system 112. The end-user application can be accessed through a network 126 (e.g., the Internet) by users of client devices, such as a personal computer 128, a smart phone 130, a tablet computer 132, and a laptop computer 124. Other client devices are possible. In alternative examples, the game data 122, the user data 123, and/or the group data 124, or any portions thereof, can be stored on one or more client devices. Additionally or alternatively, software components for the system 100 (e.g., the game module 114, the user health module 116, the group health module 118, and/or the group recommendation module 120) or any portions thereof can reside on or be used to perform operations on one or more client devices.

FIG. 1 depicts the game module 114, the user health module 116, the group health module 118, and the group recommendation module 120 as being able to communicate with the databases (e.g., the game data 122, the user data 123, and the group data 124). The game data 122 database generally includes information related to a multi-player online game implemented using the system 100. The game data 122 database can include, for example, information related to a virtual environment for the game, image, video and/or audio data for the game, event data corresponding to previous, current or future events, and/or game state data defining a current state of the game. The user data 123 database generally includes data related to user interactions with the online game and/or the virtual environment. Such information can be or include, for example, a history of user connections to the system 100, user purchases, user accomplishments, user tasks, user interactions with other users (e.g., group chats), user purchases, and/or user item usage. The group data 124 database generally includes information related to groups of users of the system 100. Such information can be or include, for example, a history of group membership, group accomplishments, group leadership (e.g., an identification of a group leader), and/or group chats.

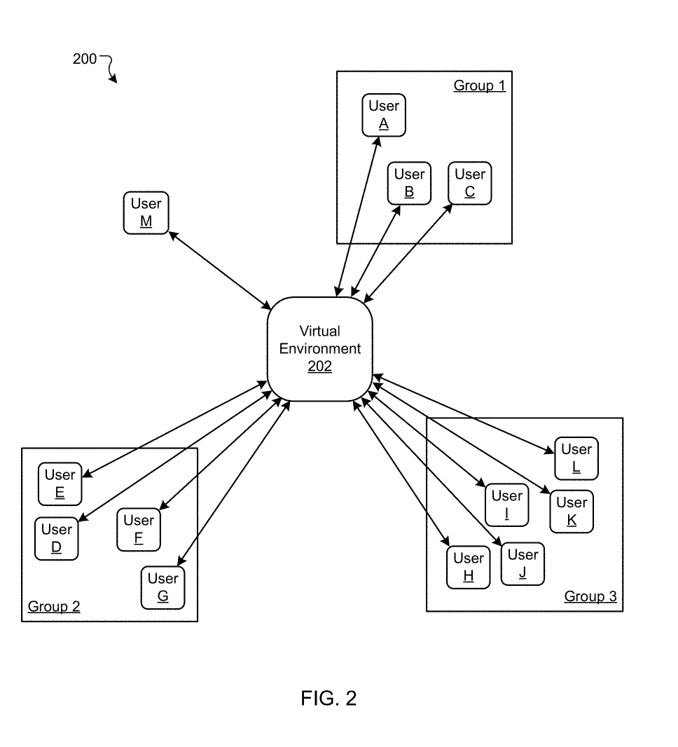

Referring to FIG. 2, in various examples, an online game 200 includes a plurality of groups of users who interact with a virtual environment 202. In the depicted example, the online game 200 includes users A-M and user groups 1-3. Group 1 includes users A, B, and C, group 2 includes users D, E, F, and G, and group 3 includes users H, I, J, K, and L. User M is not presently assigned to any group. In general, the game module 114 can implement the virtual environment 202, coordinate events, and advance the state of the game. As the users A-M and groups of users 1-3 interact with the virtual environment 202, information related to the interactions can be stored in the user data 123 database and the group data 124 database. The user health module 116 and the group health module 118 can use the user data 123 and group data 124 to calculate health scores for the users and groups. In general, a health score for a user or group provides an indication of how active the user or group is in the virtual environment 202.

In certain examples, the user health module 116 uses one or more equations and/or trained classifiers to determine health scores for the users. The one or more equations can be or include, for example, a regression equation determined by fitting a functional form (e.g., a linear or non-linear equation) through at least a portion of the user data 123. The one or more equations can receive data for a user (e.g., from user data 123 database) as input and provide a health score for the user as output. Alternatively or additionally, the one or more trained classifiers can be or include, for example, one or more linear classifiers (e.g., Fisher's linear discriminant, logistic regression, Naive Bayes classifier, and/or perceptron), support vector machines (e.g., least squares support vector machines), quadratic classifiers, kernel estimation models (e.g., k-nearest neighbor), boosting (meta-algorithm) models, decision trees (e.g., random forests), neural networks, and/or learning vector quantization models. Other classifiers can be used. In some examples, experiments performed with various algorithms indicate logistic regression classifiers can provide a best or most accurate outcome. User traits such as time spent in the game, speed at which users finish game events, and other user activity features can be used as inputs to the algorithm. The best performing input features can be chosen with feature selection algorithms where a correlation of input variables with the target variable (e.g., player health) can be used to recommend input features for selection. Once trained, the one or more classifiers can receive data for a user (e.g., from user data 123 database) as input and provide a health score for the user as output. In preferred examples, the health scores are not presented to or shared with the users.

In certain examples, various user activities in the virtual environment 202 are positive and tend to increase player health, while other user activities are negative and tend to decrease player health. Examples of positive user activities can include, for example, logging in to the online game regularly, chatting or interacting with other users, making purchases in the online game, and/or sharing items with other users. Examples of negative user activities can include, for example, not logging in to the online game for extended periods of time (e.g., several days or weeks), not chatting or interacting with other users, not making purchases in the online game, and/or not sharing items with other users. For each activity, positive or negative, the health score of a user can increase or decrease accordingly.

In one example, the user health module 116 calculates user health scores based on points associated with user actions in the online game. The health score for a user can be determined, for example, by summing the points associated with the user's actions. In general, each type of action can be associated with points that can remain constant or can decay over time. If the points for an action remain constant over time, the points can contribute a constant amount to a user's health score over successive time intervals (e.g., hours, days, weeks, or months) in the online game. Examples of actions that can be associated with constant points can include, for example, being a member of a user group and/or spending money in the online game. By contrast, if the points for an action decay over time, the points can contribute less to the user's health score over successive time intervals in the online game. Examples of actions that can be associated with points that decay over time can include, for example, logging in to the online game, chatting with other group members, and/or providing assistance to other group members. The initial number of awarded points and/or the rate of decay can depend on the type of action. Additionally or alternatively, the rate of decay can depend on the user's position in the online game and/or the user's current health score.

In some implementations, the user health module 116 uses a classification model and/or a logistic regression model, trained with data collected over a period of time for many different users of an online game. For example, Table 1 includes a summary of training data for 7,000 users who are considered to be healthy in the online game. Such healthy players can include, for example, players who have a history of active game play with little or no periods of inactive play. The table presents various statistics for the training data, include values for mean, standard deviation, minimum, 25th percentile, 50th percentile, 75th percentile, and maximum. Likewise, Table 2 includes a summary of training data for 7,000 users who are considered to be unhealthy in the online game. Such unhealthy players can include, for example, players who spend periods of time away from the game (e.g., in short durations) and/or who are not active in gameplay.

The features in the left-hand column of Tables 1 and 2 represent user activities for the online game. The features in this example can be as follows: “revenue” can be a measure of how much money each user has spent on the online game; “login_counts” can be a measure of how many times each user has logged in to the online game; “help_all” can be a measure of how many times each user has helped other users in the online game; “complete_quest” can be a measure of how many times each user completed a quest (e.g., a mission or task) in the online game; “trade” can be a measure of how many times each user traded an item with another user in the online game; “last_sleep_days” can be a measure of how long each user's last offline duration was for the online game; “chats” can be a measure of how many times each user chatted with another user in the online game; and “use_item” can be a measure of how many times each user has used an item in the online game. Other activities for generating training data are possible. In general, each of these features can represent a number of occurrences during a time period associated with the online game. For example, the revenue feature can be an amount of money spent by a player during the past day, three days, week, or month. Likewise, login_counts can be a number of times a player logged in to the game during the past day, three days, week, or month.

TABLE 1
Summary of training data for healthy users.

| Feature | count | Mean | std | min | 25% | 50% | 75% | max |
|---|---|---|---|---|---|---|---|---|
| revenue | 7000 | 145.56 | 854.84 | 0 | 0 | 0 | 24.98 | 30246.86 |
| login_counts | 7000 | 360.78 | 371.98 | 2 | 117 | 224 | 469.25 | 3864 |
| help_all | 7000 | 618.41 | 7568.55 | 0 | 55 | 182 | 545.25 | 500439 |
| complete_quest | 7000 | 2451.79 | 3928.99 | 0 | 345 | 1022 | 3042 | 70263 |
| trade | 7000 | 61.3 | 269.66 | 0 | 0 | 1 | 16 | 12265 |
| last_sleep_days | 7000 | 21.21 | 25.85 | 0.08 | 0.24 | 3.87 | 44.1 | 91.21 |
| level | 7000 | 13.47 | 4.17 | 1 | 10 | 12 | 16 | 22 |
| chats | 7000 | 529.53 | 2173.29 | 0 | 0 | 14 | 164 | 45098 |
| use_item | 7000 | 1997.81 | 3449.05 | 0 | 261 | 796 | 2241 | 62798 |

TABLE 2
Summary of training data for unhealthy users.

| Feature | count | mean | std | min | 25% | 50% | 75% | max |
|---|---|---|---|---|---|---|---|---|
| revenue | 7000 | 0.1 | 2.8 | 0 | 0 | 0 | 0 | 199.98 |
| login_counts | 7000 | 3.43 | 1.44 | 2 | 2 | 3 | 4 | 8 |
| help_all | 7000 | 0.47 | 2.91 | 0 | 0 | 0 | 0 | 70 |
| complete_quest | 7000 | 0.58 | 2.58 | 0 | 0 | 0 | 0 | 62 |
| trade | 7000 | 0.01 | 0.64 | 0 | 0 | 0 | 0 | 52 |
| last_sleep_days | 7000 | 15.65 | 9.7 | 0.09 | 8.19 | 15.21 | 23.33 | 93.77 |
| level | 7000 | 2.51 | 1.27 | 1 | 2 | 2 | 3 | 11 |
| chats | 7000 | 0.31 | 17.62 | 0 | 0 | 0 | 0 | 1465 |
| use_item | 7000 | 3.58 | 18.91 | 0 | 1 | 1 | 3 | 697 |

In certain instances, a linear functional form for a user health score H is given by

H=c₀+c₁*f₁+c₂*f₂+ . . . +cₙ*fₙ, (1)

where c₀, c₁, c₂, . . . , cₙ are constants and f₁, f₂, . . . , fₙ are features representing user activity in the online game, such as the features listed in Tables 1 and 2. In one example, when the functional form in equation (1) is fit to training data (e.g., the training data summarized in Tables 1 and 2 and/or stored in the user data 123 database) to determine values for the constants c₀, c₁, c₂, . . . , cₙ, the resulting regression equation for a user health score, H, can be as follows:

H=−3.3070+0.1166*revenue+0.3759*login_counts+0.0582*help_all+0.0395*complete_quest+0.0412*trade−0.4119*last_sleep_days+0.0032*chats+0.0155*use_item. (2)

In general, positive H values from equations (1) and (2) indicate that a user is healthy, while negative H values indicate the user is unhealthy. A highly negative H value (e.g., less than −10) can indicate (e.g., with more than 95% confidence) that a user has “churned,” which generally means the user failed to connect to the online game for more than a specified time period, such as, for example, two or three weeks or one or more months. A less negative H value (e.g., between −10 and zero) can indicate that the user is unhealthy or relatively inactive in the online game but has not churned. Such a score can indicate, for example, that the user connects to the online game periodically but does not actively participate with other users and/or spend much money on the online game. An H value greater than zero generally indicates that a user is healthy and active in the online game. When the predictions from equation (2) were compared with the original training data for active users, using cross-validation, the precision, recall, and F1 were determined to be 98.27%, 99.56%, and 98.91%, respectively.

While equation (2) is generally accurate, results from the equation indicate that active players can have excessively high health scores and that health scores for inactive players may not be equally negative. To address this issue, output from equation (2) can be adjusted or transformed to provide more balance between healthy and unhealthy scores. This can be achieved, for example, by applying a sigmoid function to positive H values and setting a hard floor for negative H values. In one example, the original health score H can be transformed to a modified health score Hₘ as follows: if H>0, then

$${H}_{m} = \frac{100}{1 + {1.001}^{- H}} - 50 ;$$

otherwise, if H>−50, then Hₘ=H; otherwise, Hₘ=−50. With this approach, −50<Hₘ<50 and, in general, only users who churned early and/or for a long time (e.g., more than a month) can have a health score near −50. The original health score H and/or the transformed health score Hₘ can be stored (e.g., in the user data 123 database) and used to determine user health in the online game.

In some examples, the health score for a user is determined as a weighted average of a current score and one or more previous scores. For example, a weighted average user health score H_{w} at a current time t can be determined from

H_{w}(t)=wₜ*Hₜ+wₜ₋₁*Hₜ₋₁+wₜ₋₂*Hₜ₋₂+ . . . +wₜ₋ₙ*Hₜ₋ₙ, (3)

where wₜ, wₜ₋₂, and wₜ₋ₙ are constants or weights, Hₜ is a current health score for the current time t (e.g., a current day), Hₜ₋₁ is a health score for a previous time t-1 (e.g., one day before the current day), Hₜ₋₂ is a health score for a previous time t-2 (e.g., two days before the current day), and Hₜ₋ₙ is a health score for a previous time t-n (e.g., n days before the current day). Hₜ, Hₜ₋₁, Hₜ₋₂, and Hₜ₋ₙ can be, or can be derived from (e.g., a combination of), H and/or Hₘ. The weights wₜ, wₜ₋₂, and wₜ₋ₙ generally decrease going from wₜ to wₜ₋ₙ, such that more recent health scores have a greater influence on the weighted average health score H_{w}. In a typical example, four weights are used and the weights wₜ, wₜ₋₁, wₜ₋₂, and wₜ₋ₙ are 0.4, 0.32, 0.2, and 0.08, respectively. Other numbers of weights and/or values for the weights are possible. With the weighted average approach, user health scores are generally more stable and can fluctuate less over time, for example, due to variations in user activity.

Once the user health scores are determined, the group health module 118 can be used to determine health scores for one or more groups of users. In various examples, a group health score G can be determined by aggregating the health scores (e.g., H, Hₘ, and/or H_{w}) for each member in the respective group. For example, the group health score G for group 2 in FIG. 2 can be, include, or be based on an average, maximum, minimum, or median of the health scores for users D, E, F, and G.

Alternatively or additionally, the group health score G for a group can be determined based on certain group actions performed by the group or members of the group. For example, the group health score G can be determined based on points awarded to the group for performing the group actions, which can be stored in the group data 124 database. In one example, the group can be awarded points for the following: +5 points per unique user login per day; +2 points per group chat message up to a threshold value and then +0.5 points per message thereafter; +10 points per member in the alliance; +15 points per task or quest completed by the group (up to a defined threshold); and +30 points when a group leader logs in during the past 24 hours. Other point values and group actions are possible. The scoring can be modified on a per-game basis. The group health score G can be a sum of the points awarded to the group during a time period (e.g., the current day). Alternatively or additionally, the group health score G can be based on a combination of the points awarded to the group and the aggregation of the health scores for each member of the group.

In various implementations, the group health score G is based on the sum of the points awarded to the group and, in general, the group health score G increases as more points are awarded to the group. In certain examples, when a group is first created, the group can be assigned an initial group health score, which can be, for example, a minimum or average (or percentage thereof) of other group health scores. The group health score G can be recalculated and/or modified periodically (e.g., hourly or daily), based on points awarded to the group.

In various examples, as with the user health scores, the group health score can be determined as a weighted average of a current score and one or more previous scores. For example, a weighted average group health score at a current time t can be determined from

G_{w}(t)=wₜ*Gₜ+wₜ₋₁*Gₜ₋₁+wₜ₋₂*Gₜ₋₂+= . . . +wₜ₋ₙ*Gₜ₋ₙ, (4)

where wₜ, wₜ₋₁, wₜ₋₂, and wₜ₋ₙ are constants or weights, Gₜ is a current group health score for the current time t (e.g., a current day), Gₜ₋₁ is a group health score for a previous time t−1 (e.g., one day before the current day), Gₜ₋₂ is a group health score for a previous time t−2 (e.g., two days before the current day), and Gₜ₋ₙ is a group health score for a previous time t−n (e.g., n days before the current day). The weights wₜ, wₜ₋₁, wₜ₋₂, and wₜ₋ₙ generally decrease going from wₜ to wₜ₋ₙ, such that more recent group health scores have a greater influence on the weighted average group health score G_{w}. In a typical example, four weights are used and the weights wₜ, wₜ₋₁, wₜ₋₂, and wₜ₋ₙ are 0.4, 0.32, 0.2, and 0.08, respectively. Other numbers of weights and/or values for the weights are possible. With the weighted average approach, the group health scores are generally more stable and can fluctuate less over time, for example, due to variations in group activity.

Table 3 presents an example in which current group health scores are used to determine a weighted average for a group over a period of 13 days. The weights wₜ, wₜ₋₂, and wₜ₋ₙ in this case are 0.4, 0.32, 0.2, and 0.08, respectively.

TABLE 3
Example group health scores.

| | | Weighted |
|---|---|---|
| | Current | Average |
| | Group | Group Health |
| Day | Health Score | Score, G_{w} |
| 1 | 250 | 250 |
| 2 | 280 | 267 |
| 3 | 300 | 282 |
| 4 | 240 | 268 |
| 5 | 290 | 275 |
| 6 | 300 | 285 |
| 7 | 110 | 217 |
| 8 | 250 | 218 |
| 9 | 280 | 238 |
| 10 | 300 | 268 |
| 11 | 240 | 268 |
| 12 | 230 | 251 |
| 13 | 300 | 266 |

In various examples, group health scores (e.g., G and/or G_{w}) are used by the group recommendation module 120 to provide recommendations to users and/or groups regarding group memberships. The group recommendation module 120 can, for example, recommend that a new user (e.g., user M in FIG. 2) join a group that is active, based on the group health score G for the active group. In certain instances, the group recommendation module 120 can identify one or more groups having desirable group health scores and can recommend that a user join one of the identified groups. The desirable group health scores can be, for example, scores that are highest among the group health scores and/or can be scores that exceed a threshold value (e.g., an average group health score or a certain percentage of a maximum group health score). Additionally or alternatively, groups can be recommended based on other factors, besides the group health score. For example, a user who speaks Japanese may be encouraged to join a group of users who speak Japanese. This can facilitate communication among group members. In some examples, group recommendations can be made according to group size limits. For example, a group that has too many members can be considered closed and may not be recommended to a user, even if the health score for the group is desirable. In various examples, a list of recommended groups can be presented to a user on the user's client device.

In various examples, when a group's health score drops below a certain threshold, group leaders (e.g., members with higher roles) and/or other group members can receive tips and/or notifications designed to help the group achieve a higher group health score. For example, group leaders can receive a tip or notification that encourages the users to take certain actions in the online game, such as, for example, adding new members to the group, chatting with members of the group, and/or performing a task in the virtual environment. Additionally or alternatively, users in unhealthy groups can be encouraged to leave the unhealthy group and/or join healthier groups. Such tips and notifications can be sent repeatedly (e.g., daily or weekly) to group members.

In some instances, group health can be improved by removing users who have low user health scores. For example, when a new user is added to a group, one or more unhealthy users (e.g., the least healthy user(s)) can be automatically removed from the group. In this way, group health can be improved by removing unhealthy users from the group and/or encouraging new or healthy members to join the group.

Advantageously, by calculating user health scores and group health scores, as described herein, users can be encouraged to join healthy groups and/or to leave unhealthy groups. When a user is associated with a healthy group, the user can be inspired to be more involved in the game. This can result in greater user satisfaction with the game, an increased number of users who play the game, and/or more revenue generated for the game.

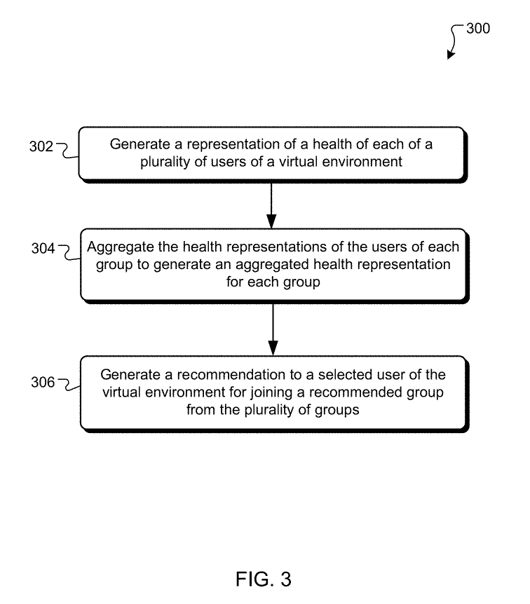

FIG. 3 illustrates an example computer-implemented method 300 of generating a recommendation for a user to join a group in an online game. A representation of a health of each of a plurality of users of a virtual environment is generated (step 302). Each health representation is preferably based on a plurality of metrics generated for a respective user from a history of interactions of the respective user with the virtual environment. Each user can be associated with one of a plurality of groups of users within the virtual environment. The health representations of the users of each group are aggregated (step 304) to generate an aggregated health representation for each group. Based on the aggregated health representations, a recommendation to a selected user of the virtual environment is generated (step 306) for joining a recommended group from the plurality of groups.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic disks, magneto-optical disks, optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including, by way of example, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse, a trackball, a touchpad, or a stylus, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what can be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing can be advantageous.

1. A method, comprising:
performing by one or more computer processors:
generating a representation of a health of each of a plurality of users of a virtual environment,
wherein each health representation is based on a plurality of metrics generated for a respective user from a history of interactions of the respective user with the virtual environment, and
wherein each user is associated with one of a plurality of groups of users within the virtual environment;
aggregating the health representations of the users of each group to generate an aggregated health representation for each group,
wherein the aggregated health representation provides an indication of how active the group is in the virtual environment;
generating, based on the aggregated health representations, a recommendation to a selected user of the virtual environment for joining a recommended group from the plurality of groups; and
adding the selected user to the recommended group.

2. The method of claim 1, wherein the health representation of each user provides an indication of how active the user is within the virtual environment.

3. The method of claim 1, wherein generating the health representation comprises:
providing the plurality of metrics to a predictive model comprising at least one of a trained classifier and a regression model.

4. The method of claim 3, wherein generating the health representations further comprises:
transforming output from the predictive model to achieve a rebalancing of the health representations.

5. The method of claim 1, wherein the plurality of metrics comprises at least one of user login activity, user chat activity, user purchasing activity, and any combination thereof.

6. The method of claim 1, wherein aggregating the health representations comprises:
determining at least one of an average, a median, a maximum, and a minimum of the health representations of the users of each group.

7. The method of claim 1, wherein generating the recommendation comprises:
determining that the recommended group comprises an aggregated health representation that exceeds the aggregated health representations of other groups within the plurality of groups.

8. The method of claim 7, wherein the recommended group comprises an aggregated health representation that is a maximum of the aggregated health representations for the plurality of groups.

9. The method of claim 1, wherein generating the recommendation comprises:
matching a language preference of the selected user with a language preference of the recommended group.

10. The method of claim 1, wherein generating the recommendation comprises:
determining that the recommended group can accommodate the selected user.

11. A system, comprising:
one or more processors programmed to perform operations comprising:
generating a representation of a health of each of a plurality of users of a virtual environment,
wherein each health representation is based on a plurality of metrics generated for a respective user from a history of interactions of the respective user with the virtual environment, and
wherein each user is associated with one of a plurality of groups of users within the virtual environment;
aggregating the health representations of the users of each group to generate an aggregated health representation for each group,
wherein the aggregated health representation provides an indication of how active the group is in the virtual environment;
generating, based on the aggregated health representations, a recommendation to a selected user of the virtual environment for joining a recommended group from the plurality of groups; and
adding the selected user to the recommended group.

12. The system of claim 11, wherein the health representation of each user provides an indication of how active the user is within the virtual environment.

13. The system of claim 11, wherein generating the health representation comprises:
providing the plurality of metrics to a predictive model comprising at least one of a trained classifier and a regression model.

14. The system of claim 13, wherein generating the health representations further comprises:
transforming output from the predictive model to achieve a rebalancing of the health representations.

15. The system of claim 11, wherein the plurality of metrics comprises at least one of user login activity, user chat activity, user purchasing activity, and any combination thereof.

16. The system of claim 11, wherein aggregating the health representations comprises:
determining at least one of an average, a median, a maximum, and a minimum of the health representations of the users of each group.

17. The system of claim 11, wherein generating the recommendation comprises:
determining that the recommended group comprises an aggregated health representation that exceeds the aggregated health representations of other groups within the plurality of groups.

18. The system of claim 17, wherein the recommended group comprises an aggregated health representation that is a maximum of the aggregated health representations for the plurality of groups.

19. The system of claim 11, wherein generating the recommendation comprises:
matching a language preference of the selected user with a language preference of the recommended group.

20. An article, comprising:
a non-transitory computer-readable medium comprising executable instructions, the executable instructions being executable by one or more processors to perform operations comprising:
generating a representation of a health of each of a plurality of users of a virtual environment,
wherein each health representation is based on a plurality of metrics generated for a respective user from a history of interactions of the respective user with the virtual environment, and
wherein each user is associated with one of a plurality of groups of users within the virtual environment;
aggregating the health representations of the users of each group to generate an aggregated health representation for each group,
wherein the aggregated health representation provides an indication of how active the group is in the virtual environment;
generating, based on the aggregated health representations, a recommendation to a selected user of the virtual environment for joining a recommended group from the plurality of groups; and
adding the selected user to the recommended group.
